(19) United States
(12) Patent Application Publication
(10) Pub. No.: US 2025/0054599 A1
(43) Pub. Date: Feb. 13, 2025

(54) SYSTEM AND METHOD FOR STANDARDIZATION OF WOUND TREATMENT GUIDELINES INFORMED BY ARTIFICIAL INTELLIGENCE

(71) Applicant: Steve Janicak, Brentwood, TN (US)

(72) Inventor: Steve Janicak, Brentwood, TN (US)

(21) Appl. No.: 18/795,317

(22) Filed: Aug. 6, 2024

Related U.S. Application Data
(60) Provisional application No. 63/531,590, filed on Aug. 9, 2023.

(51) Int. Cl. G16H 20/40; A61B 5/00; G16H 30/40

(52) U.S. Cl. CPC G16H 20/40; A61B 5/0077; A61B 5/445; A61B 5/6898; G16H 30/40

(57) ABSTRACT

The invention is a system and method for establishing an electronic-enhanced methodology for wound care assessment and treatment. Initially, a wound image is received from a mobile device, and the image is analyzed via artificial intelligence to standardize the wound area and tissue type. The wound area and tissue type analytics are then combined with point-of-care wound assessment information manually entered in a proprietary platform. The technological and point-of-care data points are then electronically combined to trigger a standardized dressing guideline with embedded evidence-based standards of care for wound treatment. The electronically generated dressing guideline is then modified over time using machine learning to identify patient and wound characteristics in combination with specific standardized dressing guidelines that result in optimal wound healing.

### CROSS-REFERENCE TO RELATED APPLICATIONS

This application claims priority to U.S. Provisional Patent Application No. 63/531,590, filed Aug. 9, 2023, the entire content of which is incorporated by reference herein.

### TECHNICAL FIELD

The presently disclosed subject matter is generally directed to a system and method for standardization of wound treatment guidelines informed by artificial intelligence.

### BACKGROUND

Chronic and complex wounds (including venous ulcers, diabetic ulcers, pressure ulcers, surgical wounds, ostomy wounds, and other complex wounds) affect millions of patients each year in the United States alone. Traditional methods and/or systems of wound care generally include manual documentation of the history of the patient, an assessment of the skin and the wound, and a determination of the likelihood of successful healing by the caregivers. As such, prevention and management of wounds are limited by the experience and knowledge of the caregivers at the particular facility or site of care. Further, errors in documentation of patient information, wound information, and/or in the management and treatment of the wound can result in a failure of the wound to heal properly. For example, the wound may be slower to heal, may worsen, or may fail to heal properly. Thus, wound care remains in its infancy with a lack of robust research to guide dressing selection.

Currently, conventional methods of wound treatment include covering the wound with a wound dressing. Conventional wound dressings are manufactured as a pre-cut sheet of multi-layer material of various shapes and sizes. The wound dressing is applied to cover the wound and a portion of the surrounding healthy skin. However, a typical wound commonly has two or more regions, including necrotic, sloughy, bacteria colonized, granulating, epithelizing, bleeding, exudating, and drying. The different wound regions typically differ by healing stage, depth, contamination, infection, and tissue stress due to patient body movement. Consequently, covering the entire wound area and surrounding healthy skin with the same dressing type may create adverse conditions for certain areas of the wound or the surrounding skin, thereby increasing the healing time.

In addition to challenges with the dressing materials, wound practitioners often employ “moist wound healing” (the industry accepted standard) to bring closure to wounds. However, the most effective wound product combinations necessary to achieve moist wound healing and thus optimal outcomes remain unknown. Utilizing standardized dressing selection guidelines rooted in evidence-based medicine provides a baseline in identifying product combinations that produce the fastest healing rates. Therefore, it would be beneficial to provide a system and method capable of coupling the standardized dressing selection guidelines with machine learning sets to incorporate patient and wound-specific data points that can interact with various wound dressing combinations to positively influence wound healing outcomes.

### SUMMARY

In some embodiments, the presently disclosed subject matter is directed to a system for optimizing wound healing in a patient. Particularly, the system comprises a mobile device configured to capture an image of the wound, wherein the wound comprise an area and a tissue type. The system further includes artificial intelligence configured to analyze the image to standardize the wound area of the wound and the tissue type of the wound. The system includes a platform for receiving the artificial intelligence analyzed image and for receiving point of care wound assessment information. The image standardization and the point-of-care wound assessment information are combined to produce a dressing guideline with evidence-based standards of care for treating the wound. The guideline is modified after a predetermined time period using machine learning to identify patient and wound characteristics in combination with the dressing guideline to optimize wound healing.

In some embodiments, the presently disclosed subject matter is directed to a method of treating a wound. Specifically, the method comprises capturing an image of the wound from a mobile device, wherein the wound comprise an area and a tissue type. The method includes analyzing the image using artificial intelligence to standardize the wound area of the wound and the tissue type of the wound via a platform. The method includes entering point-of-care wound assessment information into the platform. The method includes combining the image standardization and the point-of-care wound assessment information to produce a dressing guideline with evidence-based standards of care for treating the wound. The method includes modifying the dressing guideline after a predetermined time period using machine learning to identify patient and wound characteristics in combination with the dressing guideline to optimize wound healing.

In some embodiments, the point-of-care wound assessment information is selected from one or more of wound type, wound stage, wound depth, drainage amount, presence of purulent drainage, peri-wound characteristics, presence of undermining, and presence of tunneling.

In some embodiments, the digital device is selected from one or more of a smart phone, a smart watch, a tablet, a laptop computer, a personal digital assistant, a pair of smart glasses, a virtual reality viewing device, a digital camera, and a digital scanning device.

In some embodiments, the wound image is uploaded to a mobile application or computer for analyzing using artificial intelligence.

In some embodiments, the standardization of the wound and tissue type is determined in part by the color of the wound image.

In some embodiments, the tissue type is selected from granulation, slough, eschar, or combinations thereof.

In some embodiments, the point-of-care wound assessment information are selected from one or more of patient allergies, patient health or immune problems, topography of the body part on which the wound lies, color of skin surrounding the wound, wound depth, wound stage, drainage amount, peri-wound characteristics, presence of tunneling, and presence of undermining.

In some embodiments, the wound dressing guideline comprises a type of wound dressing and wound treatment methods.

In some embodiments, the wound dressing guideline includes physical dressing information, chemical dressing information, geometrical dressing information, optical dressing information, electrical dressing information, number of layers, porosity of a layer, thickness of a dressing, adsorbing capacity, water penetration capacity, water vapor penetration capacity, gas penetration capacity, thickness, material, material form, pharmacological or healing enhancing additives, color, local absence of dressing, adhesive, or combinations thereof.

In some embodiments, the wound dressing guideline includes predictors of non-healing by wound type.

In some embodiments, the predetermined time period is two weeks.

In some embodiments, an improvement of at least about 25% in the wound area automatically triggers a wound guideline for the wound, while an improvement of less than about 25% or deterioration in the wound area automatically triggers a non-healing wound guideline.

### BRIEF DESCRIPTION OF THE DRAWINGS

FIG. 1 is a schematic of a method of standardizing wound treatment guidelines in accordance with some embodiments of the presently disclosed subject matter.

FIG. 2a is a schematic illustrating a method of initially standardizing wound treatment guidelines in accordance with some embodiments of the presently disclosed subject matter.

FIG. 2b is a schematic illustrating a method of standardizing wound treatment guidelines at a 2 week wound recheck in accordance with some embodiments of the presently disclosed subject matter.

FIG. 2c is a schematic illustrating a method of standardizing wound treatment guidelines at a 4 week wound recheck in accordance with some embodiments of the presently disclosed subject matter.

FIG. 2d is a schematic illustrating a method of standardizing wound treatment guidelines at a 6 week wound recheck in accordance with some embodiments of the presently disclosed subject matter.

FIG. 2e is a schematic illustrating a method of standardizing wound treatment guidelines at a wound recheck in accordance with some embodiments of the presently disclosed subject matter.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

As used herein, the term “and/or” includes any and all combinations of one or more of the associated listed items.

Relative terms such as “below” or “above” or “upper” or “lower” or “horizontal” or “vertical” may be used herein to describe a relationship of one element, layer, or region to another element, layer, or region as illustrated in the drawing figures. It will be understood that these terms and those discussed above are intended to encompass different orientations of the device in addition to the orientation depicted in the drawing figures.

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

The presently disclosed subject matter is directed to a system that includes components and steps for establishing an electronic-enhanced methodology for wound care assessment and treatment. The term “wound care” refers to the application of one or more treatments to a wound to improve healing and/or prevent further damage. The term “wound” can be broadly interpreted to include any type of damage wherein the skin and/or subcutaneous tissue of a patient is torn, pierced, cut, or otherwise broken. Representative wounds can include (but are not limited to) pressure ulcers resulting from prolonged bed rest, trauma-induced wounds, diabetic ulcers, arterial insufficiency ulcers, venous stasis ulcers, burns, and the like. It should be appreciated that the disclosed system and methods can be effectively used to assess and treat a broad range of wounds and are not limited to the representative examples given above.

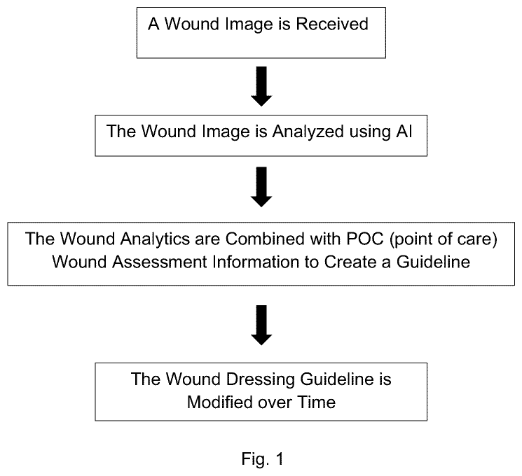

In the disclosed method, a wound image is received from a mobile device, as illustrated in the schematic of FIG. 1. The image is analyzed via artificial intelligence to standardize the wound area and tissue type. “Artificial intelligence” or “Al” refers to the development of computer systems able to perform tasks that normally require human intelligence, such as visual perception, speech recognition, decision-making, and translation between languages. “Artificial Intelligence system” refers to a system including artificial intelligence methods and/or algorithms. Thus, “artificial intelligence” refers to any suitable computer-implemented artificial intelligence technique including machine learning (supervised or unsupervised), natural language processing, machine perception, computer vision, affective computing, statistical learning and classification (including use of hidden Markov models, Bayesian network models and Kalman filters), reinforcement learning including neural networks, search algorithms and optimization algorithms (including evolutionary computing) and automated reasoning. Furthermore, although multiple computer systems and data store instances are illustrated, this is only for the purpose of illustration, and the computing environment can be enabled by a single computer system and/or data store. As used herein, the term “data store” refers to any device or combination of devices capable of storing, accessing, and/or retrieving data, which may include any combination and number of data servers, databases, data storage devices, and data storage media, in any standard, distributed, or clustered environment. In some embodiments, a data store may be housed within a computer system, or any other suitable variation.

The wound area and tissue type analytics are then combined with point-of-care wound assessment information manually entered into a proprietary platform (e.g., wound type, wound stage, wound depth, drainage amount, presence of purulent drainage, peri-wound characteristics, and/or presence of undermining or tunneling). The technological and point-of-care data points are then electronically combined to trigger a standardized dressing guideline with embedded evidence-based standards of care for wound treatment. The electronically generated dressing guideline is then modified over time using machine learning to identify patient and wound characteristics in combination with specific standardized dressing guidelines that result in optimal wound healing.

“Machine learning” refers to a type of artificial intelligence that provides processors with an ability to learn from and make predictions on data without being explicitly programmed. Machine learning focuses on the development of computer programs that can teach themselves to grow and change when exposed to new data. An algorithm for machine learning is referred to as a “machine learning algorithm”. A machine learning algorithm searches for a pattern in data used for training the machine learning algorithm and uses the pattern to detect patterns in new data and adjust program actions accordingly. The data used for training the machine learning algorithm is referred to as “training data”. Machine learning algorithms are categorized as being supervised or unsupervised. Supervised machine learning algorithms infer from the training data and apply learning to new data. Unsupervised machine learning algorithms draw inferences from the training data. Thus, machine learning includes algorithms, systems and apparatus in the field of artificial intelligence that often use statistical techniques and artificial neural networks to give computer the ability to “learn” (i.e., progressively improve performance on a specific task) from data without being explicitly programmed.

As set forth above, the disclosed system and method include an initial assessment of a wound. Particularly, a clinician can capture a digital image of a wound using any suitable device, such as a smart phone, a smart watch, a tablet, a laptop computer, a personal digital assistant, a pair of smart glasses, a virtual reality viewing device, a digital camera, a digital scanning device, and the like. The device can therefore include any apparatus capable of capturing an image. The device is used to generate a digital image of the wound and the surrounding tissue. The image can be captured by a clinician that is overseeing or involved in the care of the patient. The term “clinician” includes any type of medical personnel treating a patient, including doctors, nurses, or any other medical care provider, and may include support personnel.

The clinician (e.g., nurse, doctor, health aid, caregiver, etc.) can upload the image to a mobile application or computer for initial assessment. The computer or processor is programmed with a software program for receiving the digital image captured by the clinician (e.g., characteristics or attributes of the wound). The computer or microprocessor may be programmed to implement one or more software algorithms for achieving the functionality described in the specification and corresponding figures. In some embodiments, the mobile device can also include a communication device that is capable of storing a software application programmed for a specific operating system (e.g., iOS, Android, and Windows). The mobile device can include an electronic display, such as a graphical user interface (GUI), for providing visual images to a user, such as a clinician or patient. The mobile device may be configured to communicate with one or more networks of the therapy network. In some embodiments, the mobile device may include a cellular modem and may be configured to communicate with the network(s) through a cellular connection. In other embodiments, the mobile device may include a Bluetooth® radio or other wireless radio technology for communicating with the network(s). The mobile device can be configured to transmit data related to the tissue site of the patient.

Specifically, various characteristics of a wound can be determined using the image and the mobile application, such as (but not limited to) total wound area, wound length, wound width, wound circumference, wound volume, percentage of tissue present in the wound, wound depth, wound type, presence of purulent drainage, peri-wound characteristics, present of undermining, and/or presence of tunneling. “Total wound area” refers to the calculation of the size of a wound. The “percentage of tissue present in a wound” refers to a measurement of the amount of tissue present and/or visible within a wound. For example, healthy granulation tissue is pink or red and is a good indicator of healing, while unhealthy granulation is dark in color and can indicate the presence of wound infection. The “wound depth” is the measurement of how deep the wound is. A “wound type” identifies the type of wound (e.g., abrasions, lacerations, bites, punctures, incisions, ulcerations, burns, and the like). A wound may include chronic, acute, traumatic, subacute, and dehisced wounds, partial-thickness burns, ulcers (such as diabetic, pressure, or venous insufficiency ulcers), flaps, and grafts, for example. The term “presence of purulent drainage” refers to the presence of liquid drainage in a wound (e.g., typically of a thick consistency with a milky appearance that is often green, yellow, brown, or white in color). The term “peri-wound characteristics” refers to the appearance of the tissue surrounding a wound. The term “undermining” refers to the condition when tissue under the wound edges becomes eroded, resulting in a pocket beneath the skin at the edge of the wound. The term “tunneling” refers to a condition characterized by passageways formed underneath the surface of the skin.

In some embodiments, the wound characteristics can be determined (at least in part or fully) by analyzing the color of the wound image. In these embodiments, the percentage of wound tissue can be converted electronically in the disclosed system and method into a corresponding tissue type. For example, the tissue type can be selected from one or more of granulation (pink and/or red in color as an indication of healthy tissue in the proliferative phase of healing), slough (yellow, tan, and/or grey in color as an indication of devitalized tissue), and eschar (black in color as an indication of necrotic tissue). The predominant tissue type (e.g., the tissue type present in the largest percentage in the wound) can be used to identify the tissue type data point in the disclosed system.

Next, the wound is physically assessed by a clinician, resulting in the production of a corresponding wound dressing guideline. For example, the clinician can enter a wound type according to the wound diagnosis in the disclosed system. The evaluation process, in one embodiment, also includes an evaluation of certain characteristics of the patient. These patient characteristics may include those characteristics that could affect the success of a treatment routine for the type and severity of the wound. For example, the characteristics may include allergies, health or immune problems, topography of the body part on which the wound lies, and a color of the surrounding healthy skin. Additional characteristics can be entered by the clinician, such as (but not limited to) wound depth, wound stage, drainage amount, peri-wound characteristics, and/or presence of tunneling/undermining.

The additional wound characteristics are combined with the generated wound image data points and follow a decision tree to produce a standardized wound dressing guideline for the wound. The term “wound dressing” refers to materials placed proximal to a wound that have absorbent, adhesive, protective, osmoregulatory, pH-regulatory, and/or pressure-inducing properties. Wound dressings can be in direct or indirect contact with a wound. The recommended wound dressing guideline can specify products to apply to or adjacent to a wound and treatments to be performed on the wound. For example, application of soap and water, antibacterial ointment, non-adherent gauze, polyester fabric sheets, and the like can be recommended as part of the wound dressing guideline. In addition, the guidelines can include specific treatments, such as the frequency of cleaning the wound, frequency of changing the wound bandaging, debridement, and the like. The wound dressing guideline also includes treatment guidelines based on wound type which are evidence based and considered the standard of care in wound healing.

In one embodiment, the characteristics of the wound dressing are determined (at least in part) based on the size and shape of the areas or regions of the wound. In this embodiment, the dimensions of the wound dressing are set to match size, shape, and depth of the wound area. The materials of the wound dressing can be determined by the region of the wound (e.g., body area) and/or patient characteristics (age, general health condition, obesity level, etc.).

The defined wound dressing properties or characteristics may include physical, chemical, geometrical, optical, electrical, number of layers, porosity of a layer, thickness, and any other considerations. The determined or assigned characteristics of the wound dressing may include adsorbing capacity, water penetration capacity, water vapor penetration capacity, gas penetration capacity, thickness, material, material form (e.g., continuous film or fiber), number of layers, pharmacological or healing enhancing additives, color, local absence of dressing, and adhesive.

Other characteristics important to the healing process may also be assigned. In other words, based on the wound characteristics, a wound treatment need is determined. For example, a wound having high exudate areas requires a high absorbing and high water evaporation material dressing property. Areas having low exudates and epithelizing wound areas require low absorbing with limited water permeability material dressing property to keep wound moist environment. The healthy skin area around the wound may be used for the wound dressing attachment with, as an example, medical adhesive. Also, the portion of the wound dressing corresponding to healthy skin should be breathable and suitable for holding an adhesive. This portion of the dressing, for example, may be a porous film or fiber web that is completely permeable for gas/vapor but provides mechanical support for the dressing and attachment to the health skin.

In some embodiments, the disclosed system also includes predictors of non-healing by wound type in generating the appropriate wound dressing guideline. The predictors of non-healing can be automatically identified and are programmed based on the wound and/or determinations known to be predictive of poor or absent wound healing.

In the disclosed system, the evidence-based wound care guideline automatically generates an order in the wound the treatment plan while the treatment portion of the guideline populates in the treatment plan section of the platform. Thus, the disclosed system includes evaluating the wound, creating a determination and visual image of wound properties or characteristics, and fabricating and adapting or customizing a wound dressing and treatment protocol.

After a predetermined period of time (e.g., 2 weeks later), the wound can be reassessed by repeating the entire process. However, the evidence-based wound care guideline replaces the predictors of poor wound healing with a calculated percentage of improvement in the wound area. Specifically, the percentage of improvement in wound area can be generated automatically by the disclosed platform. It has been determined that an improvement of at least 25% in wound area is the threshold to trigger a wound guideline identified for a healing wound. Conversely, an improvement of less than 25% or a deterioration in the wound area triggers a non-healing wound guideline. Such determinations are made automatically in the disclosed platform.

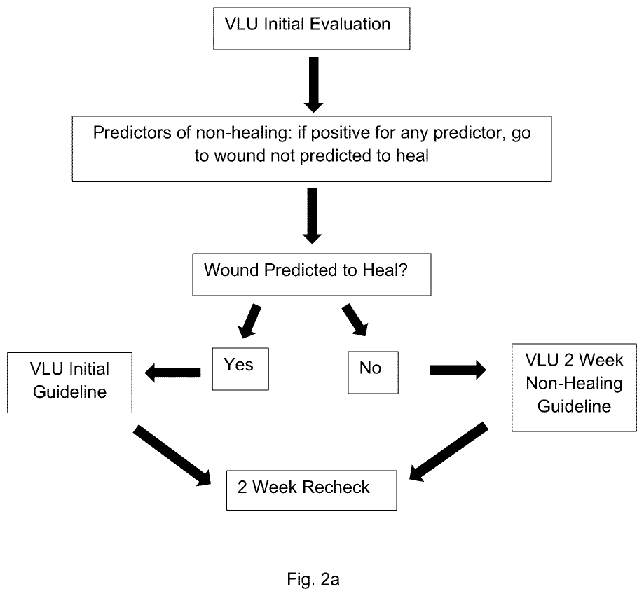

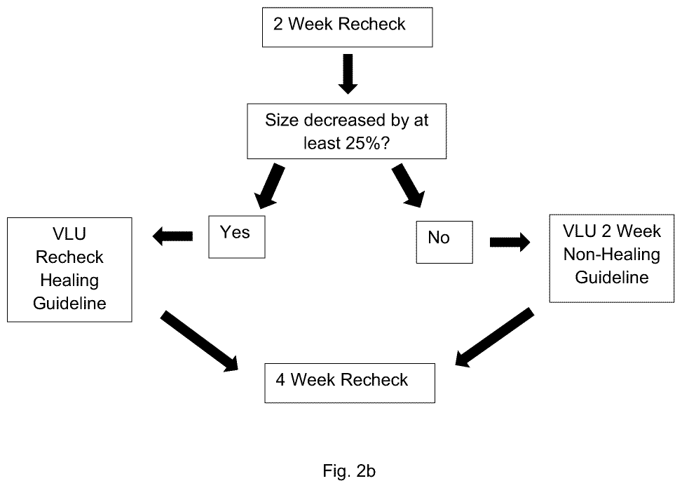

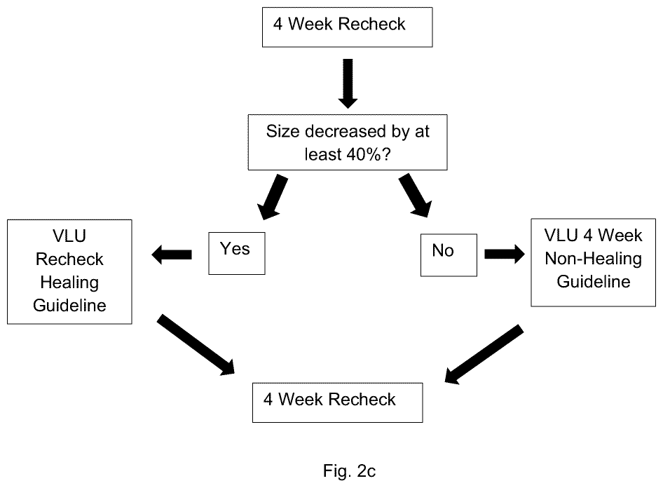

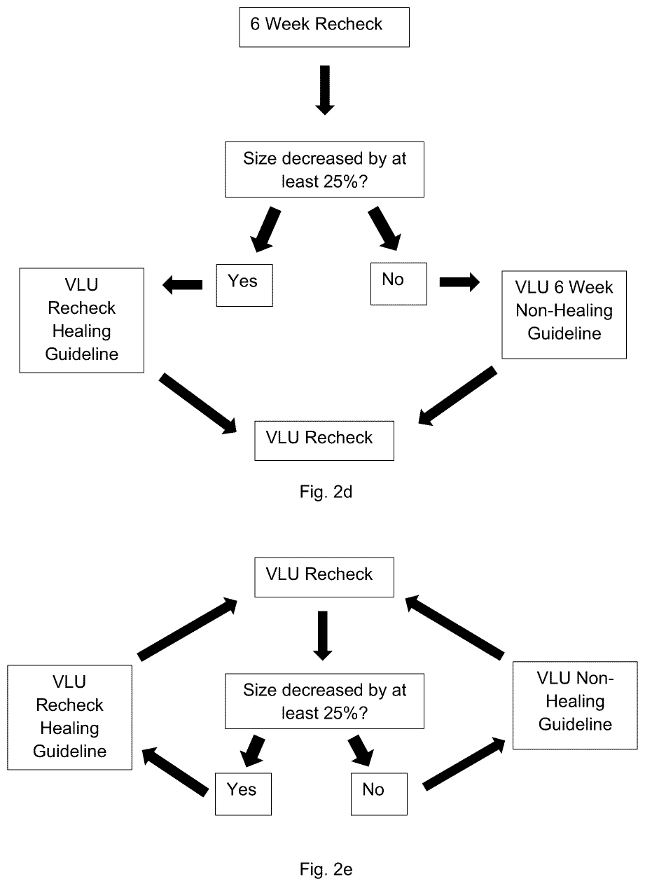

FIG. 2a is a diagram of the disclosed flow process of the disclosed system based on a venous leg ulcer (VLU) wound type. Particularly, the VLU undergoes an initial evaluation where predictors of non-healing are determined. The initial evaluation can be done by a clinician. If there are any positive predictors of non-healing, a determination that the wound is not predicted to heal is given. If the wound is not predicted to heal, an associated VLU 2-week non-healing guideline is generated. If the wound is predicted to heal, a VLU initial healing guideline is generated. It should be appreciated that the presently disclosed subject matter is not limited to 2-week time period and any predetermined time period can be used (e.g., multiple times a day, once a day, once every other day, several times a week, once a week, once every 2 weeks, once every 3 weeks, once every 4 weeks, once a month, etc.).

After the predetermined time period (e.g., 2 weeks), the wound is rechecked, as illustrated in FIG. 2b. Specifically, an assessment is performed to determine whether the wound has decreased in area by at least 25% from the initial measurement. If the wound has decreased in area by at least 25% (or any desired or set percentage, such as 5, 10, 15, 20, 25, 30, 35, 40, 45, 50 percent), the VLU recheck healing guideline is generated, and the wound is rechecked in an additional predetermined time period (e.g., 2 weeks which is 4 weeks from the initial assessment). If the wound has not decreased in area by at least 25% (or the set percentage) from the initial measurement, a VLU 2 week non-healing guideline is generated, and the wound is rechecked in an additional predetermined time period (e.g., 2 weeks which is 4 weeks from the initial assessment).

At the recheck, the wound is assessed to determine whether the wound area has decreased in area by at least 40% from the initial measurement, as illustrated in FIG. 2c. If the wound has decreased in area by at least 40%, the VLU recheck healing guideline is generated, and the wound is rechecked in an additional predetermined time period (e.g., 2 weeks which is 6 weeks from the initial assessment). If the wound has not decreased in area by at least 40% from the initial measurement, a VLU non-healing guideline is generated, and the wound is rechecked in an additional predetermined time period (e.g., 2 weeks which is 6 weeks from the initial assessment). The 40% decrease is a representative, non-limiting value that can encompass, for example, 30, 35, 40, 45, 50, 55, 60, 65, 70 or 75 percent in some embodiments.

At the recheck, the wound is assessed to determine whether the wound area has decreased by at least 25% from the prior measurement, as illustrated in FIG. 2d. If the wound has decreased in area by at least 25%, the VLU recheck healing guideline is generated, and the wound is rechecked in an additional predetermined time period (e.g., 2 weeks from the previous recheck). If the wound has not decreased in area by at least 25% from the prior measurement, a VLU non-healing guideline is generated and the wound is rechecked in an additional predetermined time period (e.g., 2 weeks from the prior recheck). As noted above, the 25% range can vary as desired and can be customized depending on patient characteristics and/or wound characteristics. The process continues for successive predetermined time intervals until the wound has healed, as shown in FIG. 2e.

Over time, the disclosed evidence-based wound care guidelines can be modified through machine learning to reflect patient and wound characteristics that combine to influence which particular dressing and treatment combinations provide optimal wound healing times. For example, the machine learning process can utilize a computer to provide dressing treatment guidelines comprising product combinations producing the best healing rates for a particular wound. Machine learning typically occurs via a computer that is not explicitly programmed but provides feedback by recognizing patterns from mined data. In such embodiments, the patterns include ongoing analysis of patient demographics, patient characteristics (such as immobility), and/or wound characteristics (such as area and drainage amount) that combine and subsequently influence which types of wound dressings and treatment plans produce the fastest wound healing.

The disclosed system and method offer many advantages over prior art systems. For example, the system and method incorporate the above components to provide a complete platform to capture, evaluate, document, and communicate clinical information for the purpose of wound prevention and treatment.

In addition, the present system is able to effectively ensure that wounds are measured uniformly. The uniformity of the system makes consulting and cross-referencing much more feasible.

The disclosed system and methods can be effectively used to improve wound care in human patients. However, the presently disclosed subject matter is not limited and the system can be used for veterinary purposes as well.

Exemplary embodiments of the methods and components of the presently disclosed subject matter have been described herein. As noted elsewhere, these embodiments have been described for illustrative purposes only, and are not limiting. Other embodiments are possible and are covered by the presently disclosed subject matter. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

What is claimed is:

1. A system for optimizing wound healing in a patient, the system comprising:
a mobile device configured to capture an image of the wound, wherein the wound comprise an area and a tissue type;
artificial intelligence configured to analyze the image to standardize the wound area of the wound and the tissue type of the wound;
a platform for receiving the artificial intelligence analyzed image and for receiving point of care wound assessment information;
wherein the image standardization and the point-of-care wound assessment information are combined to produce a dressing guideline with evidence-based standards of care for treating the wound; and
wherein the dressing guideline is modified after a predetermined time period using machine learning to identify patient and wound characteristics in combination with the dressing guideline to optimize wound healing.

2. The system of claim 1, wherein the point-of-care wound assessment information is selected from one or more of wound type, wound stage, wound depth, drainage amount, presence of purulent drainage, peri-wound characteristics, presence of undermining, and presence of tunneling.

3. The system of claim 1, wherein the digital device is selected from one or more of a smart phone, a smart watch, a tablet, a laptop computer, a personal digital assistant, a pair of smart glasses, a virtual reality viewing device, a digital camera, and a digital scanning device.

4. The system of claim 1, wherein the wound image is uploaded to a mobile application or computer for analyzing using artificial intelligence.

5. The system of claim 1, wherein the standardization of the wound and tissue type is determined in part by the color of the wound image.

6. The system of claim 1, wherein the tissue type is selected from granulation, slough, eschar, or combinations thereof.

7. The system of claim 1, wherein the point-of-care wound assessment information are selected from one or more of patient allergies, patient health or immune problems, topography of the body part on which the wound lies, color of skin surrounding the wound, wound depth, wound stage, drainage amount, peri-wound characteristics, presence of tunneling, and presence of undermining.

8. The system of claim 1, wherein the wound dressing guideline comprises a type of wound dressing and wound treatment methods.

9. The system of claim 8, wherein the wound dressing guideline includes physical dressing information, chemical dressing information, geometrical dressing information, optical dressing information, electrical dressing information, number of layers, porosity of a layer, thickness of a dressing, adsorbing capacity, water penetration capacity, water vapor penetration capacity, gas penetration capacity, thickness, material, material form, pharmacological or healing enhancing additives, color, local absence of dressing, adhesive, or combinations thereof.

10. The system of claim 1, wherein the wound dressing guideline includes predictors of non-healing by wound type.

11. The system of claim 1, wherein the predetermined time period is two weeks.

12. The system of claim 1, wherein an improvement of at least about 25% in the wound area automatically triggers a wound guideline for the wound, while an improvement of less than about 25% or deterioration in the wound area automatically triggers a non-healing wound guideline.

13. A method of treating a wound, the method comprising:
capturing an image of the wound from a mobile device, wherein the wound comprise an area and a tissue type;
analyzing the image using artificial intelligence to standardize the wound area of the wound and the tissue type of the wound via a platform;
entering point-of-care wound assessment information into the platform;
combining the image standardization and the point-of-care wound assessment information to produce a dressing guideline with evidence-based standards of care for treating the wound; and
modifying the dressing guideline after a predetermined time period using machine learning to identify patient and wound characteristics in combination with the dressing guideline to optimize wound healing.

14. The method of claim 13, wherein the point-of-care wound assessment information is selected from one or more of wound type, wound stage, wound depth, drainage amount, presence of purulent drainage, peri-wound characteristics, presence of undermining, and presence of tunneling.

15. The method of claim 13, wherein the digital device is selected from one or more of a smart phone, a smart watch, a tablet, a laptop computer, a personal digital assistant, a pair of smart glasses, a virtual reality viewing device, a digital camera, and a digital scanning device.

16. The method of claim 13, wherein the wound image is uploaded to a mobile application or computer for analyzing using artificial intelligence.

17. The method of claim 13, wherein the standardization of the wound and tissue type is determined in part by the color of the wound image.

18. The method of claim 13, wherein the tissue type is selected from granulation, slough, eschar, or combinations thereof.

19. The method of claim 13, wherein the point-of-care wound assessment information are selected from one or more of patient allergies, patient health or immune problems, topography of the body part on which the wound lies, color of skin surrounding the wound, wound depth, wound stage, drainage amount, peri-wound characteristics, presence of tunneling, and presence of undermining.

20. The method of claim 13, wherein the wound dressing guideline comprises a type of wound dressing and wound treatment methods.
